(19) United States
(12) Patent Application Publication
(10) Pub. No.: US 2006/0263302 A1
(43) Pub. Date: Nov. 23, 2006

(54) Systemic oxygenation via gastric perfusion with perfluorocarbon (PFC)

(75) Inventors: Joseph S. Friedberg, Philadelphia, PA (US); Shamus Carr, New London, CT (US)

Correspondence Address: DAVID S. RESNICK, 100 SUMMER STREET, NIXON PEABODY LLP, BOSTON, MA, 02110-2131, US

(73) Assignee: Thomas Jefferson University, Philadelphia, PA (US)

(21) Appl. No.: 11/333,805

(22) Filed: Jan. 17, 2006

Related U.S. Application Data
(60) Provisional application No. 60/644,234, filed on Jan. 14, 2005.

(51) Int. Cl. A61B 8/00; A61K 38/00

(52) U.S. Cl. 424/9.52; 514/12

(57) ABSTRACT

The present invention is based on the discovery that an increase in systemic oxygenation can be achieved by gastric perfusion of an oxygenated liquid, e.g., perfluorocarbon. Accordingly, the invention provides for a method of treating hypoxia comprising infusing an oxygenated perfluorocarbon fluid into the stomach of the patient for oxygenation throughout the body, and subsequently removing the fluid from the stomach.

### CROSS REFERENCED APPLICATIONS

This application claims the benefit under 35 U.S.C. §119(e) of U.S. provisional Patent Application No. 60/644,234, filed Jan. 14, 2005.

### FIELD OF INVENTION

The present application is directed to methods for treating hypoxia using oxygenated perfluorocarbon (PFC), which is administered by gastric perfusion.

### BACKGROUND OF THE INVENTION

Reversible pulmonary failure from processes such as adult respiratory distress syndrome (ARDS) or pneumonia, results in many deaths every year. Bioterrorism threatens to dramatically increase this problem. There is a need to augment gas exchange beyond the capabilities of mechanical ventilation.

### SUMMARY

The present invention is based on the discovery that an increase in systemic oxygenation can be achieved by gastric perfusion of an oxygenated perfluorocarbon. Accordingly, the invention provides for a method of treating hypoxia comprising infusing an oxygenated perfluorocarbon fluid into the stomach of the patient for oxygenation throughout the body, and subsequently removing the fluid from the stomach.

The methods of the invention can be used to treat hypoxia caused by any condition, e.g. any condition resulting in reversible lung failure, such as SARS infection. Examples of conditions that can be treated using methods of the invention include, but are not limited to, a condition selected from the group consisting of: a premature birth, heart surgery, lung surgery, pneumonia, Legionaires disease, toxic shock syndrome, emphysema, a lung transplant, adult respiratory distress syndrome (ARDS), infant respiratory distress syndrome (IRDS), acute lung injury (ALI), acute pulmonary embolus, SARS, and exposure to bioterror agents, e.g. anthrax.

### BRIEF DESCRIPTION OF FIGURES

FIG. 1 shows a peritoneal perfusion circuit.

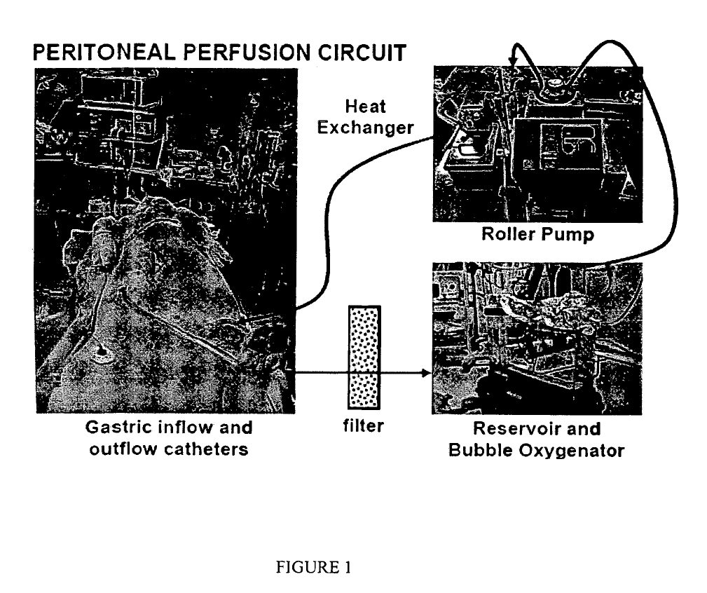

FIG. 2 shows a graph depicting the mean PaO₂ (mm Hg) (Y-axis) versus FIO₂ % (X-axis) of oxygenated versus unoxygenated saline, as decribed in Example I. Data points represent observed measurements while the lines are fitted (p-value>0.05).

FIG. 3 shows a graph depicting the mean PaO₂ (mm Hg) (Y-axis) versus FIO₂ % (X-axis) of oxygenated perfluorocarbon versus oxygenated saline as described in Example 1. Data points represent observed measurements while the lines are fitted (p-value<0.001).

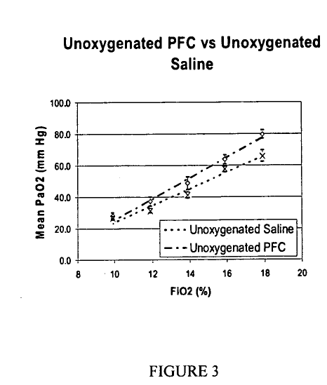

FIG. 4 shows a graph depicting the mean PaO₂ (mm Hg) (Y-axis) versus FIO₂ % (X-axis) of unoxygenated perfluorocarbon versus unoxygenated saline, as described in Example 1. Data points represent observed measurements while the lines are fitted (p-value=0.034).

### DETAILED DESCRIPTION

As used herein, the term “patient” is meant to include humans and other mammals for veterinary or research purposes, including, but not limited to, lambs, monkeys, pigs, rabbits, cats and dogs. Preferably, the patient is human.

As used herein, the term “hypoxia” refers to inadequate oxygenation of the blood. Hypoxia can arise from a variety of conditions including, but not limited to premature birth, heart surgery, lung surgery, pneumonia, Legionaires disease, toxic shock syndrome, emphysema, a lung transplant, adult respiratory distress syndrome (ARDS), infant respiratory distress syndrome (IRDS), acute lung injury (ALI) and acute pulmonary embolus.

As used herein, the term “perfluorocarbon liquid” is meant to encompass any fluorinated carbon compound with appropriate physical properties of biocompatibility. These properties are generally met by perfluorocarbons having low viscosity, low surface tension, and high solubility for oxygen and carbon dioxide making them able to readily promote gas exchange. The perfluorocarbon liquid may be made up of atoms of carbon and fluorine, or may be a fluorochemical having atoms other than just carbon and fluorine, e.g., bromine or other nonfluorine substituents. Perfluorocarbons are understood as nontoxic, fluorinated carbon compounds which are suitable for gas exchange and are thus suited for oxygenation of human or animal patients.

As used herein, the term “biocompatible” refers to materials which are generally not injurious to biological functions and which will not result in any degree of ucompatabile toxicity, icluding allergenic responses and disease states.

As used herein, the term “oxygenated perfluorocarbon liquid” refers to a specific type of gassed perfluorocarbon liquid which has been forced to absorb oxygen such that the total concentration of oxygen contained therein is greater than that present in the same liquid at atmospheric equilibrium conditions.

Representative perfluorochemicals include bis(F-alkyl) ethanes such as C₄F₉CH═CH₄CF₉ (sometimes designated “F-44E”), i-C₃F₇CH═CHC₆F₁₃ (“F-i36E”), and C₆F₁₃CH═CHC₆F₁₃ (“F-66E”);cyclic fluorocarbons, such as C₁₀F₁₈ (“F-decalin”, “perfluorodecalin” or “FDC”), F-adamantane (“FA”), F-methyladamantane (“FMA”), F-1,3-dimethyladamantane (“FDMA”), F-di-or F-trimethylbicyclo[3,3,1]nonane (“nonane”); perfluorinated amines, such as F-tripropylamine(“FTPA”) and F-tri-butylamine (“FTBA”), F-4-methyloctahydroquinolizine (“FMOQ”), F-n-methyl-decahydroisoquinoline (“FMIQ”), F-n-methyldecahydroquinoline (“FHQ”), F-n-cyclohexylpurrolidine (“FCHP”) and F-2-butyltetrahydrofuran (“FC-75” or “RM101”). Brominated perfluorocarbons include 1-bromo-heptadecafluoro-octane(C8F₁₇Br, sometimes designated perfluorooctylbromide or “PFOB”), 1-bromopenta-decafluoroheptane(C₇F₁₅Br), and I-bromotridecafluorohexane(C₆F₁₃Br, sometimes known as perfluorohexylbromide or “PFHB”). Other brominated fluorocarbons are disclosed in U.S. Pat. No. 3,975,512. Other suitable perfluorocarbons are mentioned in EP 908 178 A1.

Also contemplated are perfluorocarbons having nonfluorine substituents, such as perfluorooctyl chloride, perfluorooctyl hydride, and similar compounds having different numbers of carbon atoms.

Additional perfluorocarbons contemplated in accordance with this invention include perfluoroalkylated ethers or polyethers, such as (CF₃)₂CFO(CF₂CF₂)₂OCF(CF₃)₂, (CF₃)₂CFO—(CF₂CF₂)₃OCF(CF₃), (CF₃)CFO(CF₂CF₂)F, (CF₃)₂CFO(CF₂CF₂)₂F, (C₆F₁₃)₂O. Further, fluorocarbon-hydrocarbon compounds, include, for example, C₈F₁₇C₂H₅ and C₆F₁₃CH═CHC₆H₁₃. It will be appreciated that esters, thioethers, and other variously modified mixed fluorocarbon-hydrocarbon compounds are also encompassed within the broad definition of “fluorocarbon” liquids suitable for use in the present invention. Mixtures of fluorocarbons are also contemplated and are considered to fall within the meaning of “fluorocarbon liquids” as used herein. Additional “fluorocarbons” contemplated are those having properties that would lend themselves to gas exchange including FC-75, FC-77, RM-101, Hostinert 130, APF-145, APF-140, APF-125, perfluorodecalin, perfluorooctylbromide, perfluorobutyl-tetrahydrofuran, perfluoropropyl-tetrahydropyran, dimethyl-adamantane, trimethyl-bicyclo-nonane, and mixtures thereof.

There perfluorocarbon for use in the invention can also be in the form of an emulsion.

In one preferred embodiment, the perfluorocabon used in methods of the invention is trans-bis-perfluorobutyl ethylene (P44E).

In methods of the invention, perfluorocarbon liquid is introduced into the stomach using standard gastric perfusion techniques, for example using a catheter.

Administration can be performed continuously over a period of hours (e.g. 6-12 hours), or administered intermittently for shorter periods of time. In one preferred embodiment, the perfluorocarbon is administered continuously for 6 hours, more preferably for 2 hours. Alternatively, the oxygenated perfluorocarbon may be administered once a day or once a week for as long as necessary to ameliorate or retard the effects of the ischemic or hypoxic conditions. In other embodiments the oxygenated perfluorocarbon may be administered intermittently as needed (i.e. as determined by clinical indicators). Intermittent administration can be performed for as long as necessary, typically for a period of hours. Clinical indicators of reversed hypoxic conditions are well known to those skilled in the art.

The methods of the invention can be used to treat hypoxia that arises from any condition. For example, hypoxia is a associated with exposure to bioterror agents, premature birth, heart surgery, lung surgery, pneumonia, Legionaires disease, toxic shock syndrome, emphysema, a lung transplant, adult respiratory distress syndrome (ARDS), infant respiratory distress syndrome (IRDS), acute lung injury (ALI) and acute pulmonary embolus.

The invention can be better understood by way of the following example which is not to be construed as limiting the scope of the invention.

### EXAMPLE

Materials and Methods

Animals: Fifteen Sus scrofa pigs weighing 45-55 kg were used for the experiments. All experiments were approved by the Thomas Jefferson University Institutional Animal Care and Use Committee. The care and handling of all animals were in accord with United States Department of Agriculture guidelines.

Surgical procedure: The animals were induced with Telazol (Fort Dodge Animal Health, Fort Dodge, Iowa) 5 mg/kg injected intramuscularly (IM), after which an intravenous catheter was placed in an ear vein and 30 mg of Propofol (Baxter, Irvine, Calif.) was injected intravenously (IV). The animals were then intubated with a 7.5 mm internal diameter endotracheal tube (Mallinckrodt Inc., St. Louis, Mo.) and maintained with isoflurane inhalation anesthesia, using intermittent Propofol injections for any evidence of discomfort or awakening. The animals were placed on a Harvard Apparatus Dual Phase Control Respirator Pump (South Natick, Mass.) mechanical ventilator with a tidal volume of 10-15 ml/kg and a rate of 12-15 breaths per minute. Prior to subjecting the animal to hypoxia, the respiratory rate was occasionally adjusted in an effort to maintain a neutral pH, as determined by arterial blood gas measurements. Electrocardiography leads were placed in the standard 3-lead positions on each animal, starting at the time of induction, and continuously monitored throughout the experiment. Oxygen saturation was also measured with a Vet/Ox Plus 4700 (SDI, Waukesha, Wis.) pulse oxymeter with the probe placed on the animal's tongue. Once under general anesthesia and in the supine position, a cervical cut down was performed through which a Swan-Ganz right heart catheter (Edwards Lifesciences, Irvine, Calif.) was placed via the internal jugular vein and an 18-gauge angiocatheter was placed in the common carotid artery for access to the central arterial system for blood pressure monitoring and arterial blood gas measurements. Both of these lines were transduced using a pressure monitoring kit with a TruWave pressure transducer (Edwards Lifesciences, Irvine, Calif.) and connected to a Hewlett-Packard Monitor 78834A (Palo Alto, Calif.) system for monitoring the systemic and pulmonary arterial pressures and the central venous pressure.

After placement of the arterial and venous lines, a lower midline laparotomy was performed. A 28 French venous return cannula catheter (Edwards Lifesciences, Irvine, Calif.) was placed in the left paracolic gutter and brought out through a separate site in the left lower quadrant and sutured in place using a number 1 Prolene suture (Ethicon, Sommerville, N.J.). A 42 French venous return cannula (Edwards Lifesciences, Irvine, Calif.) was placed in an identical manner on the right side, with the tip lodged above the dome of the liver. The abdomen was then closed in a single layer with a running number I Prolene. In the first two animals in each experimental group, an additional 14 French catheter (Edwards Lifesciences, Irvine, Calif.) was placed near the midline for pressure monitoring purposes and was maintained in a vertical attitude, attached to a ruler such that the height of the column of fluid could be easily monitored. Peritoneal pressure measurements in centimeters of H₂O were calculated by multiplying column height by the correction factor 1.67 (specific gravity of trans-bis-perfluorobutyl ethylene).

Hypoxia circuit: A Matrx anesthesia machine (Orchard Park, N.Y.) was used for blending gasses and isoflurane inhaled anesthetic. Pure nitrogen, rather than filtered room air, was mixed with pure oxygen to achieve subatmospheric concentrations of oxygen. The animal was ventilated using a standard bifurcated anesthesia circuit to maintain unidirectional flow of gasses. A Datex machine (Puritan-Bennett Corporation, Overland Park, Kans.) was attached to the ventilation circuit and allowed real-time monitoring of the oxygen and anesthetic agent concentration. The oxygen and nitrogen tanks were connected to the circuit through a Matrx flow meter. The probe of the flow meter was placed at the origin of the endotracheal tube allowing for accurate determination of the respective flows of oxygen and nitrogen required to achieve the desired FiO₂.

Peritoneal circulation circuit: The 42 French venous return cannula, used to recover the perfusate from the abdomen, was connected to a 127 cm length of ⅜ inch (internal diameter) tubing (Gish Biomedical, Rancho Santa Margarita, Calif.). This tubing was connected to a #10 Clear Cold Water Filter Housing containing a 50 micron pleated filter (Filter Store, Inc., Mendon, N.Y.). This filter was used to remove any large particulate matter from entering a collecting tank and possibly clogging the circuit. The outflow of the filter drained by gravity into a custom built 10-liter glass tank (Advanced Aquatanks, Los Angeles, Calif.). The outflow of the filter was placed 41 cm below the level of the operating table. A ceramic plate diffuser (Aquaculture technology, Kitzbuhel, Austria) was placed at the bottom of the collection tank. This diffuser was designed for oxygenating ponds and gives off a steady stream of 10-200 micron bubbles when being infused with oxygen at 30 psi (based on product information from the company). This diffuser was connected to a medical oxygen tank via ¼ inch (internal diameter) tubing (Gish Biomedical, Rancho Santa Margarita, Calif.). The gauge on the tank was able to measure the oxygen delivered to the diffuser in pounds per square inch (psi). (A standard cardiopulmonary bypass “lung” cannot be used with perfluorocarbon as the liquid is able to pass through hollow fibers.)

Fluid was retrieved from the collecting tank with a 150 cm length of ¼ inch tubing that was connected to a piece of ⅜ inch tubing that passed through a Sarns Roller Blood Pump (Sams, Inc., Ann Arbor, Mich.) that was used to control flow rate. This outflow from the roller pump passed into 43 cm length of ¼ inch tubing. This tubing was connected to the inflow end of an ECMOtherm-II heat exchanger (Avecor, Minneapolis, Minn.). The heat exchanger was set to warm the abdominal perfusate to 39° C., normal body temperature for swine. This was accomplished using a Haake circulatory heater (Berlin, Germany). After being warmed by the beat exchanger, it passed through a 63 cm length of ¼ inch tubing to the 28 French inflow catheter. All tubing was standard cardiopulmonary bypass tubing.

Perfluorocarbon: Seventy liters of neat perfluorocarbon F44E (trans-bis-perfluorobutyl ethylene) were provided as a generous gift from Neuron Therapeutics, Inc (Malvern, Pa.). This perfluorocarbon is clear, colorless, odorless, immiscible with water or blood and has the physical characteristics listed in Table 1.

TABLE 1
Properties of perfluorocarbon

| | |
|---|---|
| Specific gravity | 1.668 g/cc |
| Oxygen capacity | 50 vol % |
| Carbon dioxide capacity | 200 vol % |
| Boiling point | 132° C. |

Experiments: The experimental groups included eight animals perfused with oxygenated perfluorocarbon. The controls included seven animals perfused with both oxygenated and unoxygenated saline (Table 1). In each case, the animals were stabilized at an inspired oxygen concentration (FiO₂) of 21% for 30 minutes and then sequentially dropped to an FiO2 of 18%, 16%, 14%, 12% and 10% for 30 minutes at each level. The desired FiO₂ was achieved by empirically adjusting the blend of nitrogen and oxygen to achieve the desired FiO₂ as indicated by the in-line Datex gas monitor. The initial descending oxygenation phase of each experiment was conducted without activating the peritoneal perfusion circuit. Arterial and mixed venous blood gasses, along with heart rate and blood pressure, central venous pressure, and pulmonary arterial pressures were obtained at each level. Blood gases were performed using a Gem Premier 3000 (Instrumentation Laboratory, Lexington, Mass.). For the first two experiments in each group, the intraperitoneal pressure was also recorded, both before and during peritoneal perfusion.

At the conclusion of the descending FiO₂ phase, the peritoneal circuit was activated. The flow rate of the inflow was adjusted such that it was equal to the gravity drainage flow rate from the outflow catheter. This rate was empirically determined and unique for each animal, falling between 1.2 and 4.2 liters/minute (Table 1). The amount of perfusate that was placed in the system was also idiosyncratic and was adjusted to maintain approximately six to seven liters in the reservoir. The ascending FiO₂ levels were achieved by resuming the same nitrogen and oxygen flow settings that had been used for the descending FiO₂ phase. The animals were again allowed to equilibrate for 30 minutes during the ascending phase prior to collecting blood gases. The oxygen supply to the bubbler in each case was the same at 30 pounds/square inch.

Results

Table 2 summarizes the groups of animals that received either oxygenated saline or perfluorocarbon as the perfusate.

TABLE 2
Experimental variables

| | A. Perfusion Medium | | |
|---|---|---|---|
| | Saline | perfluorocarbon | |
| B. VARIABLE | (N = 7) | (N = 8) | p-value |
| GENDER, n (%) | | | 1.0 |
| Male | 1 (14) | 1 (13) | |
| Female | 6 (86) | 7 (87) | |
| WEIGHT (kg), mean (sd) | 49.4 ± 2.8 | 48.6 ± 2.1 | 0.51 |
| FLOW RATE (L/min), | 2.0 ± 0.9 | 2.6 ± 1.2 | 0.28 |
| mean (sd) | | | |
| VOLUME in PERITONEAL | 2.5 ± 0.4 | 2.7 ± 0.3 | 0.34 |
| CAVITY (L), mean (sd) | | | |
| TOTAL VOLUME (L), mean | 8.4 ± 1.7 | 9.1 ± .4 | 0.33 |
| (sd) | | | |

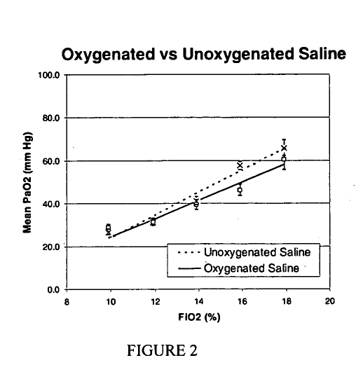

The study included eight pigs in the experimental group (perfluorocarbon) and seven pigs in the control (saline) group. All animals underwent the same experimental protocol with the exception of what liquid was used to perfuse the peritoneum. The measured intraperitoneal pressures for steady state perfusion were less than 15 cm of water. During the experiments these animals were observed at five different levels of FiO₂ (18%, 16%, 14%, 12%, and 10%) under two different conditions (oxygenation off or on). A total of 132 measurements were obtained, 69 when oxygenation was off and 63 when oxygenation was on. Table 3 presents the observed and fitted PaO₂ means for the two groups, by oxygenation condition and FiO2. For unoxygenated saline, with the circuit off, as the FiO₂ was lowered from 18% to 10%, the mean PaO₂ decreased from 65.9±9.7 mm Hg to 26.6±2.8 mmHg, respectively. This provided fitted values of 66.7 mm Hg (95% CI=61.0 to 72.5) to 22.7 mm Hg (95% CI=17.0 to 28.3), respectively. These were considered the baseline values against which all other measurements were compared. After obtaining data for the animal at an FiO₂ of 10%, the circuit was started. As the animal was raised from a FiO₂ of 10% to 18%, the PaO₂ increased from 28.6±3.9 mm Hg to 60.5±11.6 mmHg, respectively. This resulted in fitted data 25.6 mm Hg (95% CI=20.1 to 31.1) to 55.9 mm Hg (95% CI=49.0 to 62.7), respectively. There was no significant difference between the values obtained from the animals when the saline was oxygenated or not oxygenated (FIG. 2: p-value >0.05).

TABLE 3
Observed and fitted PaO₂ means for the saline and
perfluorocarbon(perfluorocarbon) groups, by oxygenation condition and FiO₂.

| | | Saline | | | | | perfluorocarbon | | | | |
|---|---|---|---|---|---|---|---|---|---|---|---|
| | | | Observed | | Fitted | | | Observed | | Fitted | |
| FiO2 (%) | O2 | n | mean | (std) | mean | 95% CI) | n | mean | (std) | mean | (95% CI) |
| 10 | Off | 5 | 26.6 | (2.8) | 22.7 | (17.0, 28.3) | 7 | 28.0 | (5.3) | 25.7 | (20.6, 30.7) |
| 12 | | 6 | 31.3 | (3.3) | 33.7 | (29.0, 38.4) | 8 | 37.1 | (8.1) | 38.5 | (34.3, 42.7) |
| 14 | | 6 | 41.2 | (4.5) | 44.7 | (40.4, 49.1) | 8 | 48.8 | (11.4) | 51.3 | (47.3, 55.3) |
| 16 | | 7 | 57.6 | (4.9) | 55.7 | (51.0, 60.5) | 7 | 63.7 | (7.9) | 64.2 | (59.7, 68.6) |
| 18 | | 7 | 65.9 | (9.7) | 66.7 | (61.0, 72.5) | 8 | 79.8 | (7.7) | 77.0 | (71.6, 82.4) |
| 10 | On | 5 | 28.6 | (3.9) | 25.6 | (20.1, 31.1) | 7 | 36.7 | (8.4) | 35.2 | (30.2, 40.1) |
| 12 | | 6 | 31.3 | (4.1) | 33.2 | (28.9, 37.4) | 8 | 43.8 | (4.8) | 44.5 | (40.8, 48.3) |
| 14 | | 5 | 39.4 | (5.0) | 40.7 | (36.6, 44.8) | 7 | 55.3 | (7.6) | 53.9 | (50.2, 57.6) |
| 16 | | 5 | 46.2 | (5.8) | 48.3 | (43.2, 53.4) | 7 | 64.4 | (8.2) | 63.3 | (58.5, 68.1) |
| 18 | | 6 | 60.5 | (11.6) | 55.9 | (49.0, 62.7) | 7 | 68.7 | (10.4) | 72.7 | (66.2, 79.2) |

Oxygenated saline was then compared to oxygenated perfluorocarbon. The data from animals that had oxygenated saline was compared directly to those who underwent perfusion with oxygenated perfluorocarbon. As the animals were raised from a FiO₂ of 10% to 18% with the circuit rurning with oxygenated perfluorocarbon the PaO₂ increased from 36.7±8.4 mm Hg to 68.7±10.4 mmHg, respectively. This resulted in fitted data of 35.2 mm Hg (95% CI=30.2 to 40.1) to 72.7 mm Hg (95% CI=66.2 to 79.2), respectively. The average increase in PaO₂ with oxygenated perfluorocarbon perfusion ranged from 8.1 to 18.2 mmHg. These differences represent the perfluorocarbon treatment effect when the perfluorocarbon was oxygenated. All five differences are significant; there is a general trend towards larger differences with increasing FiO₂ but it is not statistically significant (p=0.105). A common treatment effect can be estimated across all FiO₂ values, representing the average mean difference in oxygen uptake between perfluorocarbon and saline when oxygenation is on, irrespective of the level of FiO₂ (Table 4). This average was 12.8 mmHg (95% CI=7.4 to 18.2; p-value<0.001).

TABLE 4
Observed and estimated treatment effect: mean PaO₂ difference
between perfluorocarbon versus saline, with oxygenation on,
for different FiO₂ values.

| | |
|---|---|
| Observed | Fitted |

| FiO2 (%) | Effect | Effect | (95% CI) | p |
|---|---|---|---|---|
| 10 | 8.1 | 9.6 | (3.3, 15.9) | .005 |
| 12 | 12.4 | 11.4 | (5.9, 16.9) | .001 |
| 14 | 15.9 | 13.2 | (7.8, 18.6) | .001 |
| 16 | 18.2 | 15.0 | (8.9, 21.1) | .001 |
| 18 | 8.2 | 16.8 | (9.4, 24.3) | .001 |
| Overall | | 12.8 | (7.4, 18.2) | .001 |

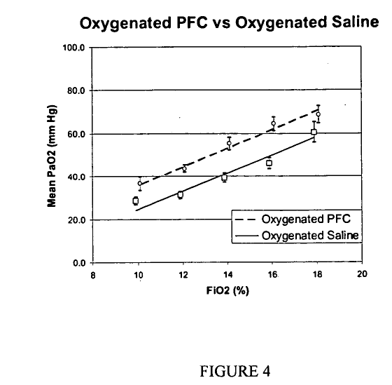

The above difference between perfluorocarbon and saline when oxygenation is on should be put in context of the same difference when oxygenation is off (FIG. 4, differences between solid and dashed lines). As mentioned above, when oxygenation was on, oxygen uptake was significantly higher in the perfluorocarbon group by about 12.8 mmHg on average. When oxygenation was off, oxygen uptake was also significantly higher in the perfluorocarbon group but by only half of that amount, i.e., 6.4 mmHg on average (95% CI=0.6 to 12.2; p-value=0.034). (This effect was thought to represent an oxygen “reservoir effect” from the indwelling perfluorocarbon. This corresponds to a positive group-by-oxygenation interaction effect of about 6.4 mmHg on average (95% CI=−1.2 to 14.0; p-value=0.095).

The most clinically relevant increase in PaO₂ came at a FiO₂ of 14%. The observed effect of oxygenated perfluorocarbon compared to oxygenated saline demonstrated an increase of 15.9 mmHg with a fitted effect of 13.2 mm Hg (95% CI=7.8, 18.6 mm Hg). This resulted in a baseline PaO₂ of 39.4±5.0 mmHg, increasing to 55.3±7.6 mmHg with oxygenated perfluorocarbon perfusion. This corresponds to an increase in arterial oxygen saturation (SaO₂) from 73% with oxygenated saline to 89% with oxygenated perfluorocarbon. None of the control experiments with oxygenated saline demonstrated any significant gas exchange effects. No statistically significant difference was noted in the arterial or venous partial pressure of carbon dioxide (PaCO₂ or PvCO₂) between groups irrespective of FiO₂ or whether saline or perfluorocarbon was used as the perfusate.

These experiments represent the first demonstration of the ability to perform peritoneal perfluorocarbon perfusion in a large animal and that this technique results in augmentation of systemic arterial oxygenation oxygen levels. Small animal studies have previously demonstrated the ability to use the peritoneal surface to deliver oxygen using intraperitoneal gaseous oxygen, oxygenated saline or oxygenated perfluorocarbon, but promising results have not previously been reported in any large animal studies [1-4]. The relative ease with which oxygenation can be increased in small, but not large, animal models is likely a function of the significantly increased ratio of the peritoneal surface to body mass in small animals [5]. Based upon our results we hypothesize that a perfusion circuit, to maintain a very high oxygen diffusion gradient, that circulates perfluorocarbon, with its extraordinary gas solubility properties, is necessary to achieve significant gas exchange through the peritoneal surface of a large animal.

The measured augmentation of systemic oxygen levels by perfusing the peritoneum with oxygenated perfluorocarbon was uniform and statistically significant for all five tested concentrations of inspired oxygen. It was the use of perfluorocarbon that resulted in the observed phenomenon, in our model, as saline failed to serve as an effective gas transport medium.

Our model achieved hypoxia by ventilating the animals with subatmospheric concentrations of oxygen. We designed this model to allow us to explore the effect of perfusion over a wide range of hypoxia. In order to specifically study the effects on oxygenation, we maintained the animals in a normocarbic state and, thus, cannot draw any conclusions regarding the impact of peritoneal perfluorocarbon perfusion to clear carbon dioxide. One could speculate, however, that the greater diffusivity of carbon dioxide compared to oxygen, and very high solubility of carbon dioxide in perfluorocarbon, would favor carbon dioxide clearance in a hypercarbic state.

In considering peritoneal perfusion as a potential treatment for patients with reversible pulmonary failure, arterial oxygen levels in the 40 mmHg range might represent the point at which a clinician might entertain instituting heroic measures for appropriate patients. This degree of hypoxia was achieved in our model at the inspired oxygen concentration of 14% which resulted in a mean arterial oxygen concentration of 39.4 mmHg, an arterial oxygen saturation of 73%. Institution of peritoneal perfusion with oxygenated perfluorocarbon resulted in an average increase of 15.9 mmHg to achieve an average arterial oxygen level of 55.3 mmHg, an arterial oxygen saturation of 89%. Although our experiments were short term, it is worth noting that this degree of augmentation in oxygenation could represent a potentially life saving measure in a patient dying from reversible pulmonary failure. The amount of oxygen delivery we observed is significantly less than can be achieved with direct blood interface techniques, like extracorporeal membrane oxygenation. Peritoneal perfusion, however, would not require anticoagulation and this is a potentially significant advantage. For example, a trauma patient with an intracranial hemorrhage and pulmonary contusions or ARDS may require extrapulmonary oxygen delivery, but would not be a candidate for any modality requiring anticoagulation.

If further research reveals that this modality remains as innocuous for prolonged periods as we observed for these short term proof of principle experiments, then there could be other roles for this modality beyond salvage of a patient in terminal pulmonary failure. One such application would be peritoneal perfusion for reversible intestinal ischemia. As the intestinal peritoneum constitutes a significant fraction of the peritoneal surface, it stands to reason that oxygen levels in ischemic bowel could be increased by bathing them in oxygenated perfluorocarbon. Another application could be prophylactic institution of this technique to avoid ventilator induced lung injury [6]. As a non-pulmonary technique for gas exchange, it could allow the clinician to decrease the toxic ventilator settings that are sometimes required to support a patient dying from potentially reversible lung failure. Such a technique could short circuit the Catch-22 that results when high ventilator settings exacerbate the underlying lung dysfunction and mandate even higher ventilator settings, further worsening the dysfunction.

The results from these experiments demonstrate significant increases in systemic oxygen levels can be achieved in a large animal induced hypoxia model by perfusing the peritoneal surface with oxygenated perfluorocarbon. This technique is useful for the supportive care of patients with profound, but reversible, pulmonary failure. This technique is also useful for support of patients with reversible bowel ischemia or early institution for a patient with potentially reversible pulmonary failure to avoid ventilator induced lung injury.

The references cited herein and throughout the application are incorporated by reference.

### REFERENCES

1. Klein J, Faithfull N S, Salt P J, et al.: Transperitoneal oxygenation with fluorocarbons. Anesth Analg 1986, 65:734-738.
2. Schmidt J A, Bilge F H, Colacino J M, et al.: Peritoneal oxygenation of normoxic and hypoxic dogs. ASAIO Transactions 1989, 35(1):35-39.
3. Giffin D M, Gow K W, Warriner C B, et al: Oxygen uptake during peritoneal ventilation in aporcine model of hypoxemia. Critical Care Medicine 1998, 26(9):1564-1568.
4. Faithfull N S, Klein J, van der Zee H T, et al.: Whole body oxygenation using intraperitoneal perfusion of fluorocarbons. BJA: British Journal of Anaesthesia 1984, 56(8):867-872.
5. Siriwardhana S A, Newfield A M, Lipton J M, et al: Oxygen delivery by the peritoneal route. Canadian Journal of Anaesthesia 1990, 37(4 Pt 2):S159.
6. Barr J, Livne A, Lushkov G, et al.: Peritoneal ventilation: An animal model of extrapulmonary ventilation in experimental adult respiratory distress syndrome. Pediatric Research 1994, 35(6):682-684.

1. A method for treating hypoxia in a patient comprising the steps of:
i. infusing an oxygenated perfluorocarbon liquid into the stomach of the patient for oxygenation throughout the body; and
ii. subsequently removing the fluid from the stomach.

2. The method of claim 1, wherein said hypoxia is due to a condition that results in reversible lung failure.

3. The method of claim 1, wherein said hypoxia is due to a condition selected from the group consisting of: exposure to a bioterror agent, premature birth, heart surgery, lung surgery, pneumonia, Legionaires disease, toxic shock syndrome, emphysema, a lung transplant, adult respiratory distress syndrome (ARDS), infant respiratory distress syndrome (IRDS), acute lung injury (ALI), SARS infection, and acute pulmonary embolus.

4. The method of claim 1, wherein the perfluorocarbon is selected from the group consisting of C₄F₉CH═CH₄CF₉, i-C₃F₇CH═CHC₆F₁₃, C₆F₁₃CH═CHC₆F₁₃, C₁₀F₁₈, perfluorodecalin, F-adamantane, F-methyladamantane, F-1,3-dimethyladamantane, F-di-or F-trimethylbicyclo[3,3,1]nonane, F-tripropylamine, F-tri-butylamine, F-4-methyloctahydroquinolizine, F-n-methyl-decahydroisoquinoline, F-n-methyldecahydroquinoline, F-n-cyclohexylpurrolidine, F-2-butyltetrahydrofuran, 1-bromo-heptadecafluoro-octane (C₈F₁₇Br), 1-bromopenta-decafluoroheptane (C₇F₁₅Br), 1-bromotridecafluorohexane(C₆F₁₃Br), (CF₃)₂CFO(CF₂CF₂)₂OCF(CF₃)₂, (CF₃)₂CFO—(CF₂CF₂)₃OCF(CF₃), (CF₃)CFO(CF₂CF₂)F, (CF₃)₂CFO(CF₂CF₂)₂F, (C₆F₁₃)₂O, C₈F₁₇C₂H₅ and C₆F₁₃CH═CHC₆H₁₃.
